(19) United States
(12) Patent Application Publication
(10) Pub. No.: US 2014/0200458 A1
(43) Pub. Date: Jul. 17, 2014

(54) THREE DIMENSIONAL METAMATERIALS FROM CONFORMAL POLYMER COATING LAYERS

(71) Applicants: Sameer Sonkusale, Arlington, MA (US); Willie John Padilla, Newton, MA (US); Mehmet Dokmeci, Brookline, MA (US)

(72) Inventors: Sameer Sonkusale, Arlington, MA (US); Willie John Padilla, Newton, MA (US); Mehmet Dokmeci, Brookline, MA (US)

(73) Assignees: Tufts University, Medford, MA (US); Northeastern University, Boston, MA (US); Trustees of Boston College, Chestnut Hill, MA (US)

(21) Appl. No.: 13/977,939

(22) PCT Filed: Dec. 22, 2011
(86) PCT No.: PCT/US11/66722

§ 371 Date: Mar. 12, 2014

Related U.S. Application Data
(60) Provisional application No. 61/429,318, filed on Jan. 3, 2011.

(51) Int. Cl. A61B 5/00; B29C 39/00

(52) U.S. Cl. CPC A61B 5/0086; B29C 39/006
USPC 600/473; 428/201; 264/1.7

(57) ABSTRACT

A manufacture for supporting propagation of terahertz waves includes a stack of layers made of a conformal protective polymer coating material; and an array of cells patterned on each of said layers, each cell including a metallic structure.

### CROSS REFERENCE TO RELATED APPLICATIONS

This application claims priority to U.S. Provisional Application No. 61/429,318, filed on Jan. 3, 2011, the contents of which are incorporated herein.

### FIELD OF DISCLOSURE

This disclosure relates to electromagnetic structures that control wave propagation, and in particular, to metamaterials for supporting such propagation in the terahertz, far-infrared and millimeter-wave range.

### BACKGROUND

Terahertz radiation is useful for a variety of applications. For example, because of its ability to penetrate most clothing, terahertz radiation provides a way to detect concealed weapons. Because of its ability to detect differences in water content and density of tissue, terahertz radiation can be used to reliably distinguish between normal cells and cancerous cells.

Electromagnetic metamaterials for supporting propagation of a particular wavelength consist of composites having metallic structures consisting of large number of unit cells each having dimensions an order smaller than the wavelength to be propagated. The joint interaction of these metallic structures in their surrounding medium results in a wave propagation medium that can have selected values of permittivity and/or permeability. Different values of permittivity/permeability can provide a diverse array of electromagnetic response such as filtering, focusing, negative reflection or refraction, lenses, cloaking and radiation.

### SUMMARY

In one aspect, the invention features a manufacture for supporting and altering propagation of terahertz and far-infrared electromagnetic waves. Such a manufacture includes a stack of layers made of a conformal protective polymer coating material; and an array of metamaterial unit cells patterned on each of the layers. Each such metamaterial unit cell includes a metallic structure

Among the embodiments of the manufacture are those in which the stack of layers includes a parylene layer, those in which the stack of layers includes a parylene-C layer, those in which the stack of layers includes a parylene-N layer, those in which the stack of layers includes a parylene-D layer, those in which the layers are biocompatible, and those in which the stack of layers includes a poly para xylene layer. Also included are those embodiments in which the layers are made of any combination of the foregoing materials

In some embodiments, the metallic structures have a maximum lineal dimension that can range anywhere from nanometers to meters. A maximum lineal dimension in the range from 100 nm to 10 mm is suitable for terahertz and far-infrared region of electromagnetic spectrum.

In another aspect, the invention features a method of making a metamaterial for propagation of terahertz waves. Such a method includes vapor coating, onto a platform, a plurality of layers of conformal protective coating material; patterning metallic planar structures on each of the layers; and removing the plurality of layers from the platform.

In some practices, the conformal coating material is a parylene.

In another aspect, the invention features an apparatus for sub epithelial implantation for detection of skin cancer using terahertz radiation. Such an apparatus includes a detector having a metamaterial, the metamaterial including layers of a biocompatible conformal protective coating polymer, each of which has, formed thereon, an array of metallic structures.

In some embodiments, the metallic structures have a maximum lineal dimension of between 0.1 mm and 1 mm. In other embodiments, the coating includes parylene.

These and other features of the invention will be apparent from the following detailed description and the accompanying figures, in which:

### DESCRIPTION OF THE FIGURES

FIG. 1 shows a multi-layer parylene-based meta-material;

FIG. 2 shows one layer of a parylene-based metamaterial of FIG. 1; and

FIG. 3 shows exemplary metallic structures for the cells of the metamaterial shown in FIG. 1.

### DETAILED DESCRIPTION

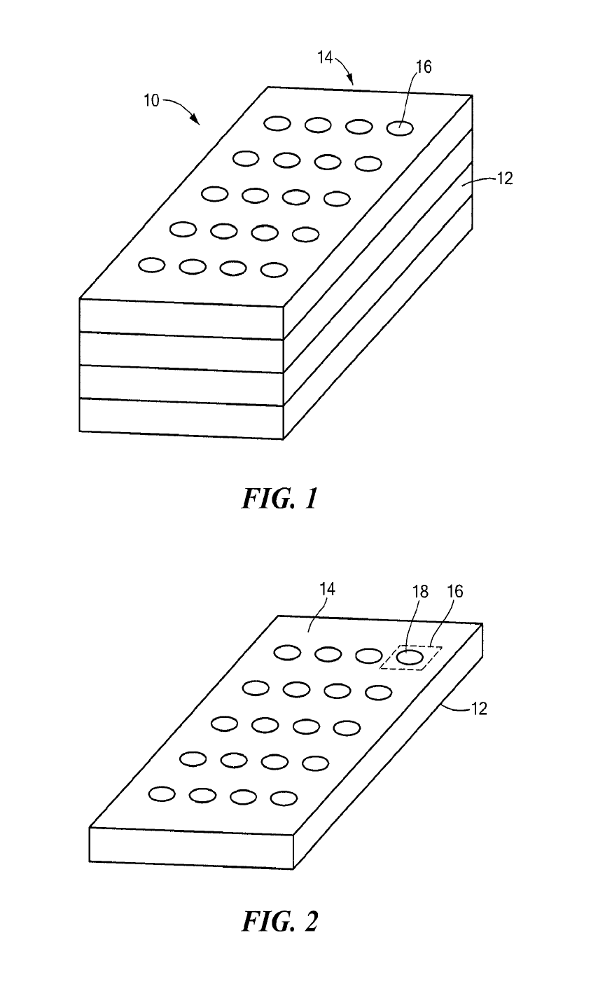

Referring to FIG. 1, a metamaterial 10 consists of a plurality of thin film layers 12, each of which is between about 10 nm and 1 mm thick. A typical layer 12, as shown in FIG. 2, has, patterned thereon, an array 14 of metamaterial unit cells 16. Each cell 16 includes a metallic sub-wavelength structure 18. In many embodiments, the thin film thickness is approximately 100 nm.

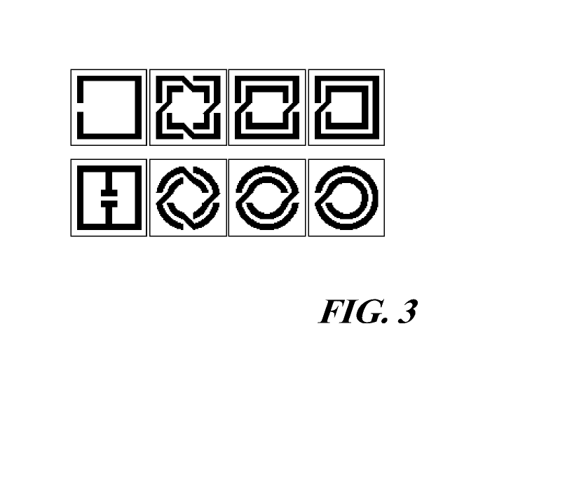

In the particular cell 16 shown, the metallic sub-wavelength structure 18 is a planar split-ring resonator. However, other metallic sub-wavelength structures can be used. For example, instead of a split-ring resonator as shown, the cell 16 can have a split-ring structure with single and/or multiple loops, or a fishnet structure, or an arrangement of thin wires. In some embodiments, the metamaterial unit cell 16 can include magneto dielectric spheres. Examples of different metallic structures in a 100 μm×100 μm cell are shown in FIG. 3.

Each layer 12 is made of a conformal protective polymer coating material. A suitable polymer is a poly para xylene parylene, and in particular, parylene-C, parylene-N, and parylene-D.

In one method of fabrication, a silicon layer is used as a platform upon which the parylene layer 12 is fabricated and from which it is peeled off after fabrication.

After dehydration baking at 150 C, a ten micron layer 12 of parylene-C is deposited onto the platform using a parylene deposition unit. A suitable deposition unit is sold under the name of LABCOPTER 2 PARYLENE DEPOSITION UNIT made by Specialty Coating Systems in Indianapolis, Ind.

The deposition unit vaporizes a dimer charge at 175 C and 1 Ton, and then decomposes it into its monomer (paraxylylene) at 690 C and 0.5 Ton. It then deposits the monomer onto the platform at 25 C and 0.1 Ton to form the parylene-C layer 12.

Once the layer 12 is in place, the next step is to create the array 14 of unit cells 16. This is carried out using a conventional photo resist, such as AZ nLOF 2020 using conventional photolithographic methods. A layer of titanium and/or gold, or any suitable conductor, is then sputtered onto the parylene layer to form the metallic sub-wavelength structures 18. The thickness of the conductor ranges from 10 nm to 200 nm. The platform, now supporting one meta-material layer 12, is then placed in an acetone bath and peeled off.

To manufacture a laminated structure as shown in FIG. 1, one patterns a layer 12 and then carries out chemical vapor deposition on the patterned layer 12 to form a second layer, which can then be patterned in the same way as the first layer. This procedure repeats until the requisite number of layers is reached.

Because of its biocompatibility, a metamaterial 10 made of parylene thin films is particularly suitable for medical applications. Because of their ability to interact with terahertz radiation, and because of the use of terahertz radiation in detecting skin cancer, diagnostic detectors that rely on parylene-based metamaterials can safely be implanted in a human.

Various properties of metamaterials as described herein are described in more detail in an article entitled “Metamaterials on parylene thin film substrates: Design, fabrication and characterization at terahertz frequency” by X. Liu, et al., and published in Applied Physics Letters 96-011906, the contents of which are herein incorporated by reference.

Having described the invention, and a preferred embodiment thereof, what we claim as new and secured by Letters Patent is:

1. A manufacture for supporting electromagnetic wave propagation, and altering propagation, said manufacture comprising:
a stack of layers made of a conformal protective polymer coating material; and
an array of meta material unit cells patterned on each of said layers, each meta material unit cell including a metallic structure.

2. The manufacture of claim 1, wherein said stack of layers comprises a parylene layer.

3. The manufacture of claim 1, wherein said stack of layers comprises a parylene-C layer.

4. The manufacture of claim 1, wherein said stack of layers comprises a parylene-N layer.

5. The manufacture of claim 1, wherein said stack of layers comprises a parylene-D layer.

6. The manufacture of claim 1, wherein said layers are biocompatible.

7. The manufacture of claim 1, wherein said stack of layers comprises a poly para xylene layer.

8. The manufacture of claim 1, wherein said metallic structures have a maximum lineal dimension between 100 nm and 10 mm.

9. A method of making a metamaterial for propagation of terahertz and millimeter waves, said method comprising:
vapor coating, onto said platform, a plurality of layers of conformal protective coating material;
patterning metallic planar structures on each of said layers;
removing said plurality of layers from said platform.

10. The method of claim 9, further comprising selecting said conformal coating material to be a parylene.

11. An apparatus for sub epithelial implantation for detection of skin cancer using terahertz radiation, said apparatus comprising:
a detector having a metamaterial, said metamaterial including layers of a biocompatible conformal protective coating polymer, each of which has, formed thereon, an array of metallic structures.

12. The apparatus of claim 11, wherein said metallic structures have a maximum lineal dimension of between 0.1 mm and 1 mm.

13. The apparatus of claim 11, wherein said coating comprises parylene.
